(19) United States
(12) Patent Application Publication
(10) Pub. No.: US 2023/0338731 A1
(43) Pub. Date: Oct. 26, 2023

(54) INTEGRATION OF ELECTROPHYSIOLOGY MAPPING SYSTEMS WITH ELECTROPORATION SYNCHRONIZED WITH PACING

(71) Applicant: Northwestern University, Evanston, IL (US)

(72) Inventors: David A. Johnson, Romeoville, IL (US); Justin K. Ng, Evanston, IL (US); Rishi K. Arora, Chicago, IL (US)

(21) Appl. No.: 18/028,934

(22) PCT Filed: Oct. 7, 2021
(86) PCT No.: PCT/US21/53912

§ 371 Date: Mar. 28, 2023

Related U.S. Application Data
(60) Provisional application No. 63/088,829, filed on Oct. 7, 2020.

(51) Int. Cl. A61N 1/32; A61N 1/372; A61N 1/04

(52) U.S. Cl. CPC A61N 1/327; A61N 1/372; A61N 1/0476; A61N 1/37211

(57) ABSTRACT

A system for performing integrated mapping and electroporation includes a basket catheter that includes a plurality of splines and a plurality of electrodes mounted to each spline in the plurality of splines. The system also includes a controller device connected to the basket catheter. The controller device includes a processor configured to activate at least a subset of electrodes to perform electroporation, where activation of at least the subset of electrodes generates current paths between electrodes on a given spline and between electrodes on adjacent splines.

### CROSS-REFERENCE TO RELATED APPLICATION

The present application claims the priority benefit of U.S. Provisional Patent App. No. 63/088,829 filed on Oct. 7, 2020, the entire disclosure of which is incorporated herein by reference.

### REFERENCE TO GOVERNMENT RIGHTS

This invention was made with government support under HL119810 awarded by the National Institutes of Health (NIH), under HL140061 awarded by the NIH, and under HL125881 awarded by the NIH. The government has certain rights in the invention.

### BACKGROUND

Electroporation, which can also be referred to as electropermeabilization, refers to a technique in the field of microbiology that enables one to introduce a substance into a cell. Specifically, traditional electroporation applies an electrical field to cells. The applied electrical field is used to increase the permeability of the cell membrane, which allows for introduction of the substance through the membrane without the use of a syringe or other device to puncture the cell membrane. The introduced substance can include chemicals, drugs, DNA, etc. for use in treatment or therapy.

### SUMMARY

An illustrative system for performing integrated mapping and electroporation includes a basket catheter that includes a plurality of splines and a plurality of electrodes mounted to each spline in the plurality of splines. The system also includes a controller device connected to the basket catheter. The controller device includes a processor configured to activate at least a subset of electrodes to perform electroporation, where activation of at least the subset of electrodes generates current paths between electrodes on a given spline and between electrodes on adjacent splines.

In an illustrative embodiment, the plurality of electrodes alternate between a positive electrode and a negative electrode along a length of a spline. In some embodiments, the controller device is further configured to receive a pacing signal for a heart that is in contact with the basket catheter and synchronize the electroporation with the pacing signal to prevent ventricle defibrillation. In other embodiments, the system includes a pacing unit that is in communication with the controller device, and the pacing signal is received from the pacing unit.

In another embodiment, the system includes an electroporation generator that is in communication with the controller device and connected to the basket catheter. In such an embodiment, the controller device can control the electroporation generator to deliver voltages to the subset of electrodes. In one embodiment, the controller device is further configured to receive mapping data from the subset of electrodes. In another embodiment, the controller device is configured to alternate the basket catheter between a mapping mode to obtain the mapping data from the subset of electrodes and an electroporation mode to perform the electroporation by the subset of electrodes. In another embodiment, the controller device further includes a display, and the mapping data is placed onto the display for viewing by a physician or other system user.

In one embodiment, the controller device is further configured to control agent delivery in conjunction with the electroporation. The agent delivery can be in-vivo delivery of one or more genes into a heart of a patient. In other embodiments, the controller device is further configured to determine whether the electroporation is successful. The controller device can also be configured to maintain a count of successful electroporations using the basket catheter. In another embodiment, at least the subset of electrodes comprises all of the electrodes on the basket catheter to maximize a coverage area of the basket catheter. Activation of the subset of electrodes can be performed sequentially such that one or more first electrodes is activated prior to one or more second electrodes. Activation of the subset of electrodes can also be performed simultaneously. In some embodiments, the plurality of electrodes are bi-polar, and the controller device is configured to set a polarity of each electrode as negative or positive.

An illustrative basket catheter for use in performing integrated mapping and electroporation includes a sheath, and a plurality of splines mounted to the sheath. The basket catheter also includes a plurality of electrodes mounted to each spline in the plurality of splines. The plurality of electrodes alternate between a positive electrode and a negative electrode along a length of each spline, and the plurality of splines are configured such that current flow occurs both between electrodes along a given spline and between electrodes on adjacent splines.

In an illustrative embodiment, each electrode in the plurality of electrodes is bi-polar such that each electrode can transition to a positive electrode or a negative electrode. In another embodiment, he plurality of splines comprises eight splines, and the plurality of electrodes mounted to each spline comprises eight electrodes. In some embodiments, a spacing between the plurality of electrodes mounted to each spline is seven millimeters. In another illustrative embodiment, each of the electrodes in the plurality of electrodes is individually wired such that each electrode can be controlled independent of any other electrode. In another embodiment, each of the electrodes is configured for placement into a mapping mode and an electroporation mode.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts a controller device for a mapping, electroporation, and pacing system (or system) in accordance with an illustrative embodiment.

FIG. 2A depicts a first portion of the schematic of the controller device in accordance with an illustrative embodiment.

FIG. 2B depicts a second portion of the schematic of the controller device in accordance with an illustrative embodiment.

FIG. 2C depicts a third portion of the schematic of the controller device in accordance with an illustrative embodiment.

FIG. 2D depicts a fourth portion of the schematic of the controller device in accordance with an illustrative embodiment.

FIG. 2E depicts a fifth portion of the schematic of the controller device in accordance with an illustrative embodiment.

FIG. 3 depicts a basket catheter having a plurality of electrodes positioned on a plurality of splines in accordance with an illustrative embodiment.

FIG. 4A shows the wiring for catheter splines A, B, and C, and part of spline D in accordance with an illustrative embodiment.

FIG. 4B shows the wiring for the remaining portion of catheter spline D, catheter splines E, F, and G, and part of spline H in accordance with an illustrative embodiment.

FIG. 4C shows the wiring for the remaining portion of catheter spline H in accordance with an illustrative embodiment.

FIG. 5 depicts a computing system in direct or indirect communication with a network in accordance with an illustrative embodiment.

FIG. 6A depicts a pulse train and total current (in Amperes) delivered by an electroporation generator to a catheter across all electrodes in accordance with an illustrative embodiment.

FIG. 6B depicts a pulse from the pulse train of FIG. 6A in accordance with an illustrative embodiment.

FIG. 6C depicts the current in a single electrode on a spline of the catheter in accordance with an illustrative embodiment.

FIG. 6D depicts an electrogram taken prior to electroporation using the proposed system in accordance with an illustrative embodiment.

FIG. 6E depicts an electrogram taken subsequent to electroporation using the proposed system in accordance with an illustrative embodiment.

### DETAILED DESCRIPTION

Recent advancements in the treatment of atrial fibrillation allow for electroporation of the heart. Electroporation refers to a process in which an electrical field is applied to a cell wall to increase the permeability of the cell wall such that a substance (i.e., agent) can be delivered into the cell. As an example, U.S. patent application Ser. No. 16/773,540 is entitled ‘Materials and Methods for Gene Delivery in the Heart’ and directed to electroporation. The entire disclosure of U.S. patent application Ser. No. 16/773,540 is incorporated by reference herein. While the embodiments described herein are primarily with respect to treatment and monitoring of the heart, it is to be understood that the proposed methods and systems are not so limited. Rather, the proposed methods and systems can be used to monitor and/or treat any organ/tissue within a patient.

In some embodiments, the methods and systems described herein can be used to electroporate a target coronary tissue such that gene/biologic (or other agent) delivery can be performed in-vivio in both large and small tissue areas of the heart or other region of interest. The electroporation can be performed prior to delivery of a desired agent, concurrently with delivery of the agent, and/or subsequent to delivery of the agent to the target coronary tissue. In some embodiments, the electroporation can be performed less than 1 hour prior to delivery of the agent. As one example, electroporation can be performed less than 1 hour, less than 45 minutes, less than 30 minutes, less than 15 minutes, less than 1 minute, etc. prior to delivery of the agent. In embodiments where the electroporation is performed following delivery of the agent, the electroporation can be performed less than 1 minute, less than 5 minutes, less than 15 minutes, less than 30 minutes, less than 45 minutes, less than 1 hour, etc. following delivery of the agent. As noted above, the electroporation can also be performed concurrently with the delivery of the agent.

The electroporation can be performed any suitable number of times for any suitable duration to achieve the desired effect. For example, electroporation may be performed once or a plurality of times, depending on the desired treatment and the effect of previously delivered agents. The electroporation may be performed via an endocardial procedure or an epicardial procedure, depending on the desired treatment for the patient. In an illustrative embodiment, the electroporation is performed via a custom multipolar basket catheter that facilitates endocardial electroporation. As discussed in more detail below, the basket catheter is designed to cover the entire surface area of a single atrium such that any portion of the atrium (or the entire atrium) can be targeted for electroporation. Such a system allows selective gene transfer to occur in the entire atrial region(s) where the electroporation is being performed.

In some embodiments, the proposed system can use the process of Low Voltage—Long pulse width Electroporation (LVEP) to deliver DNA molecules, genes, biologics, and/or other agents into (e.g., heart) cells of viable tissue. In one embodiment, the LVEP amplitudes can range from −1 Volt (V) to −500 V with pulse durations ranging from −1 milliseconds (mS) to −500 mS. It is known that the LVEP pulses can potentially generate Joule heating, which has the potential of injuring the patient. However, the system uses a short pulse duration such that any temperature rise in the wires/electrodes of the catheter can be regarded as negligible with respect to causing membrane pore creation. Scientific and engineering literature uses units of Volts/centimeter (V/cm) when discussing the electrical field effect of LVEP, and those units will be used herein.

While the aforementioned voltage field is being delivered to a region within the atrium, it can interfere with ventricular beats of the heart. However, the inventors have determined that these complications can be prevented by synchronizing the electroporation with the QRS complex by pacing it with a known source. Any type of heart pacing system known in the art may be used. Thus, in some embodiments, to avoid ventricular fibrillation (VF), the controller device can control the catheter such that the electroporation occurs during the electrocardiogram (ECG) QRS complex. The controller device can control the timing of the electroporation relative to the QRS complex by monitoring a pacing of the ventricle and using the pacing to establish a known rhythm. The controller device can do this by generating a pacing trigger signal to drive a pacing system that has an external triggered input, and/or by measuring the established pacing rate using the catheter.

In another illustrative embodiment, the proposed methods and systems can also be used to perform mapping and/or monitoring of the heart or other tissue upon which the system is being used. As an example, the proposed catheter can be used to measure electrical activity in the myocardium to assess the viability of the tissue. In some embodiments, the proposed catheter is used in conjunction with a controller device that has multiple functions, including the ability to selectively control the electrical connections of the catheter, and to allow switching between electroporation and using the catheter to obtain mapping/monitoring data.

To perform the mapping, in one embodiment, the controller device can use the basket catheter to obtain timing data regarding heart activity. In another embodiment, the controller device can connect to one or more electrophysiologic mapping catheters that work in conjunction with the basket catheter. In such an implementation, the controller device allows for switching the catheter connection from an electrophysiologic mapping system (e.g. GE CardioLab Prucka, Boston Scientific RYTHMIA Mdx, the Abbott EnSite system, etc.) to connect to electroporation systems (e.g. LeyRoy Biotech ELECTROCEL b15, Harvard Apparatus BTX 630/830, etc.). In one embodiment, the catheter used can have interelectrode spacing of 7.0 millimeters (mm). Typical delivery of the catheter can be 200 V/7 mm (roughly 28.6V/mm) with a pulse width of 10 mS and 10 pulses delivered in the range of 500 to 1000 mS. Other electrode spacing, pulse widths, and pulse rates may be used in alternative embodiments. Similarly, a different catheter configuration and/or electroporation system may be used.

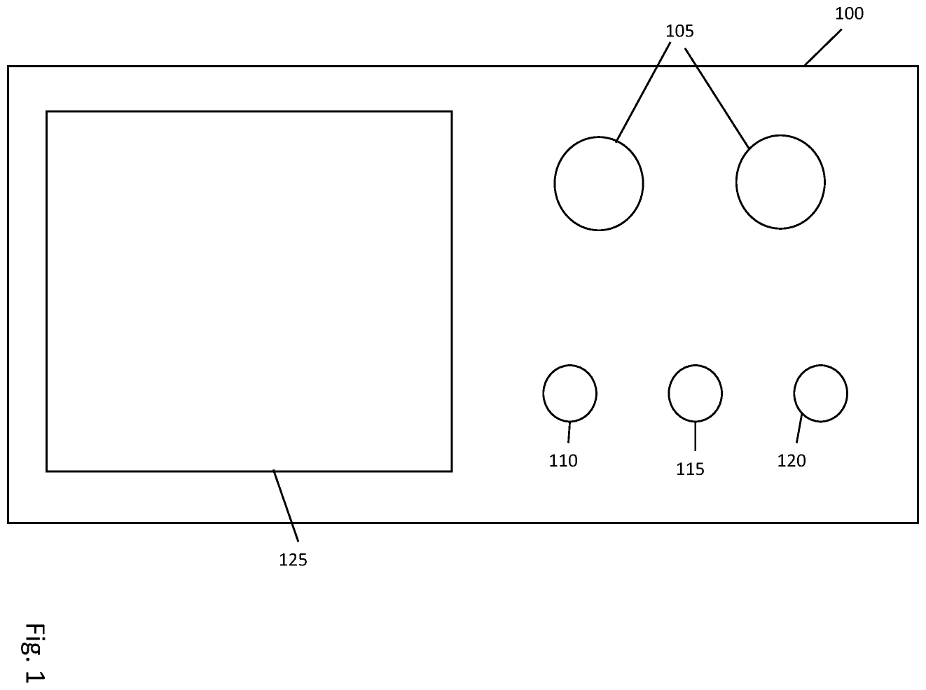

FIG. 1 depicts a controller device 100 for a mapping, electroporation, and pacing system (or system) in accordance with an illustrative embodiment. The controller device 100 includes catheter ports 105 that are used to send control signals to the electrodes of one or more catheters and receive data from the one or more catheters. While 2 catheter ports 105 are shown, it is to be understood that the controller device 100 can include fewer or additional catheter ports 105, depending on the implementation. An illustrative basket catheter to be used with the system is described with reference to FIG. 3.

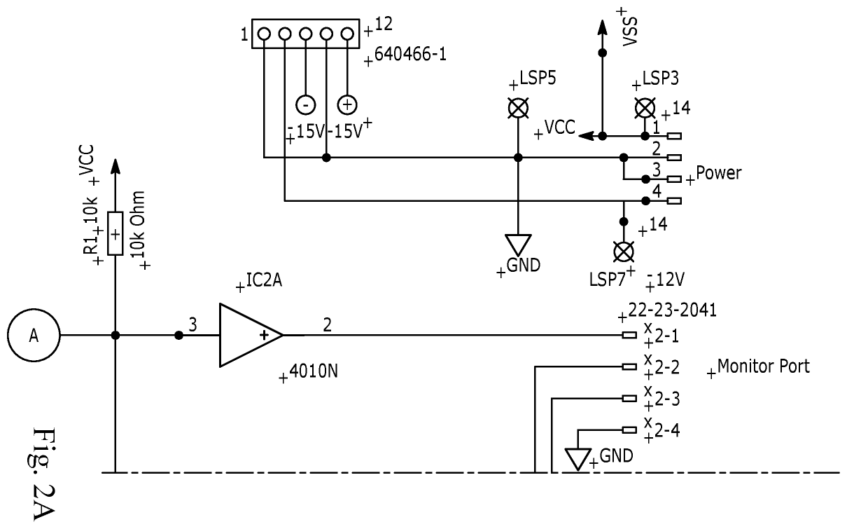

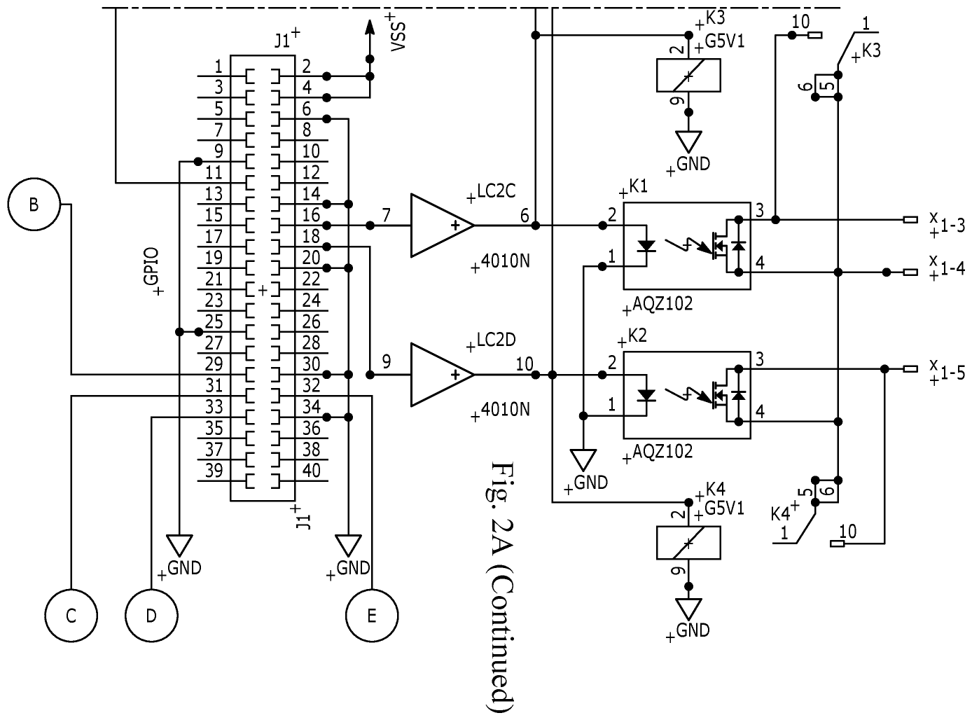

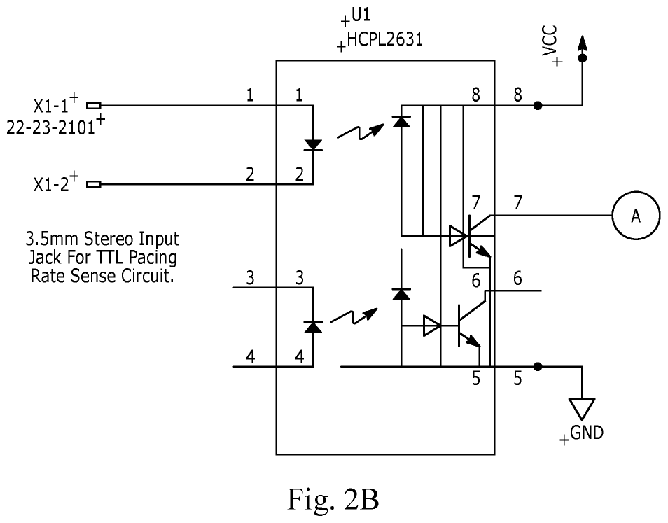

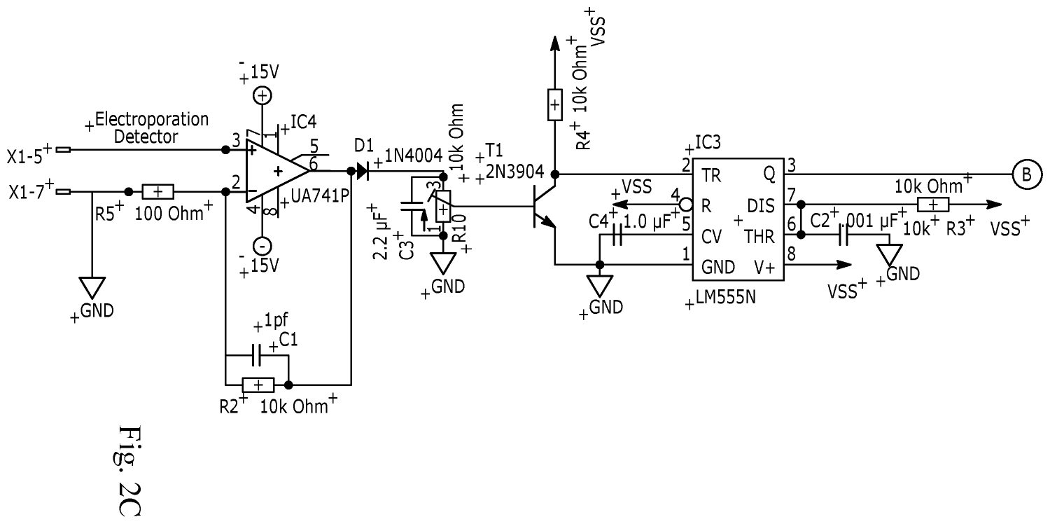

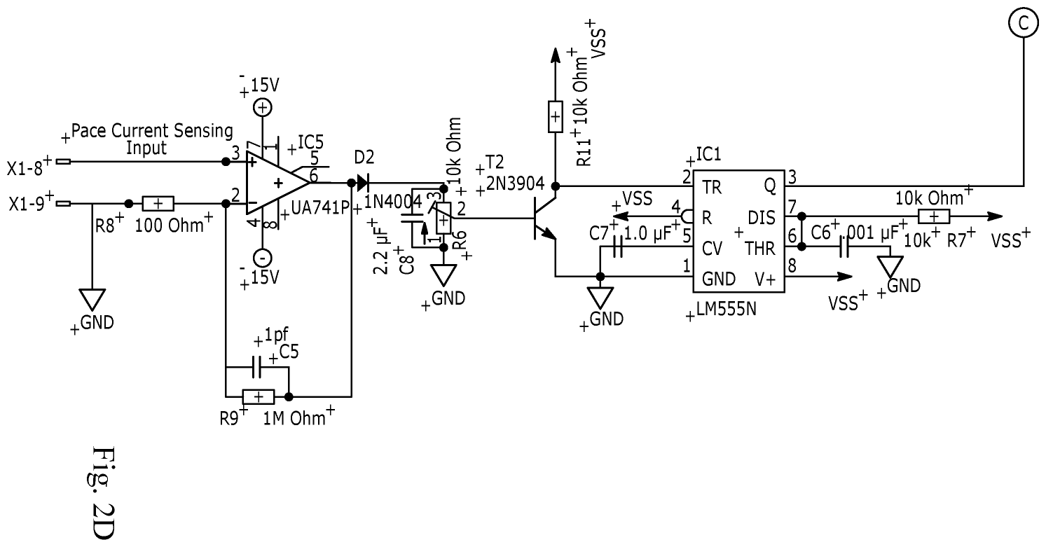

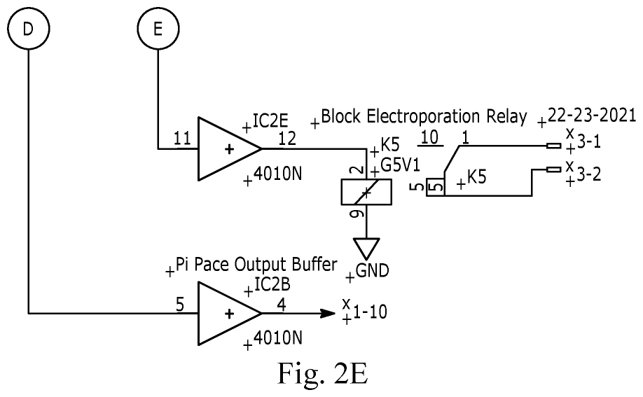

The controller device 100 also includes a pacing input 110, a foot switch input 115, and an electroporator input 120. The pacing input 110 is used in some embodiments to receive a signal from a pacing device that is used to help monitor cardiac pace. In one embodiment, the pacing input 110 can be in the form of a ⅛ inch jack that receives a transistor-transistor logic (TTL) input from an opto-isolator (e.g., an HP2631 opto-isolator) that provides isolation from the input to the internal integrated computer system [TICS]. The IICS can run a custom C program that the inventors developed. Alternatively, a different programming language may be used. This program has an option to measure the TTL pacing rate, and provides signaling via isolation relays (e.g., K1-K4 in FIG. 2) to charge and discharge an external electroporation generator. In an alternative embodiment, the user can select a pacing rate and have the IICS provide a TTL signal to drive an external pacing stimulus system. As a result, the electroporation can be synchronized with an external input such as a paced heartbeat to help prevent ventricle defibrillation.

The foot switch input 115 can receive an input from a foot-activated switch that the user can press to switch between system modes, send control signals, etc. For example, the foot-activated switch can be used to switch the controller device 100 from an electroporation mode to a mapping mode. Any other control/functionality of the system can similarly be controlled by the foot-activated switch in alternative embodiments. In alternative embodiments, the foot switch input 115 may be replaced by or used in conjunction with a hand operated switch on the controller device 100 that performs the same function(s).

The electroporator input 120 can be used to detect the current delivered by an electroporation generator. The electroporation generator can be incorporated into the controller device 100, or implemented as a standalone device that is in communication with the controller device 100 via the electroporator input 120. The controller device 100 also allows a user to configure a catheter for electroporation, and the controller device 100 can further be used to detect electroporation. If electroporation is successful, the controller device 100 can increment a counter that is used to keep track of successful electroporations.

The controller device 100 also includes a display 125 that can be a liquid crystal display (LCD), light-emitting diode (LED) display, or any other type of computer display screen known in the art. The display 125 can be a touchscreen display in one embodiment such that the user is able to control the system, manipulate the display, etc. through touch. In an illustrative embodiment, the display 125 is used to display operating instructions for the system, data received from the basket catheter, data received from a pacing device, data related to electroporation monitoring, including a count of successful electroporations, electroporation control settings such as pulse length, voltage value, etc., mapping data, etc.

In an illustrative embodiment, the computer system integrated into the controller device 100 can include a memory upon which custom software is stored. The custom software can be used to enable multiple functionalities for the system. For example, the user can select a maximum number of electroporation pulses to deliver. The user can configure the software to measure the rate of an external pacing generator. Similarly, the user can input a pacing rate that causes the controller device 100 to generate an external sync pulse to drive an external pacing system. The software can also be used to control the charge of an external electroporation generator. In an illustrative embodiment, the software times the delivery of the electroporation to occur during the pace (e.g. QRS) beat to avoid VF. The software and internal integrated custom hardware can also be used to deliver genes/biologics or other agents during the electroporation process. The software and internal integrated custom hardware is also used to detect successful electroporation delivery of the gene or other agent by detection of electroporation current changes. As discussed above, any of the control settings, received data, etc. can be presented to a user through the display 125. The user can make selections through a touch screen functionality of the display 125 and/or one or more controls (e.g., buttons, switches, etc.) can be included on the housing of the controller device 100.

In another illustrative embodiment, the controller device 100 can be configured to analyze variations in mapping data (e.g., Topera FirmMap data) to determine system parameters to be used during electroporation. The controller device 100 can also determine system parameters in the form of variables that are based on the outcome of dose response data that is received by the controller device 100. As a hypothetical example, if one expects an effective energy density of 200V/.7CM @10 mS pulse, then in a smaller basket one can use fewer electrodes to maintain that density, or the voltage can be decreased to maintain the same density.

FIG. 2 depicts a complete schematic of a controller device 100 in accordance with an illustrative embodiment. Due to size, the schematic has been divided into several subsections. Specifically, FIG. 2A depicts a first portion of the schematic of the controller device 100 in accordance with an illustrative embodiment. FIG. 2B depicts a second portion of the schematic of the controller device 100 in accordance with an illustrative embodiment. FIG. 2C depicts a third portion of the schematic of the controller device 100 in accordance with an illustrative embodiment. FIG. 2D depicts a fourth portion of the schematic of the controller device 100 in accordance with an illustrative embodiment. FIG. 2E depicts a fifth portion of the schematic of the controller device 100 in accordance with an illustrative embodiment.

Capital letters in circles are used to denote how the circuit portions of FIGS. 2A-2E connect to one another. Specifically, the circled capital A indicates how the circuit portion depicted in FIG. 2B connects to the circuit portion depicted in FIG. 2A. The circled capital B indicates how the circuit portion depicted in FIG. 2C connects to the circuit portion depicted in FIG. 2A. The circled capital C indicates how the circuit portion depicted in FIG. 2D connects to the circuit portion depicted in FIG. 2A. The circled capital letters D and E indicates how the circuit portion depicted in FIG. 2E connects to the circuit portion depicted in FIG. 2A.

Still referring to FIG. 2, in an illustrative embodiment, all connections to the catheter are each wired to a Double Pole, Double Throw [DPDT] rated greater than both a common LVEP and an expected amperage to be used for successful LVEP. Each DPDT relay can include two Normally Connected [NC] poles, two Normally Open [NO] poles, and two throws. As used herein, throw 1, NC 1, and NO 1 refer to the first half of the DPDT relay, and throw 2, NC 2, and NO 2 refer to the second half of the DPDT relay.

In another illustrative embodiment, an electrode on the catheter input connector is connected to a relay ‘throw 1’, and its default state NC 1 state is wired to another connector making the electrode available for external connection to an electrophysiologic mapping System (e.g. GE CardioLab, Boston Scientific Rhythmia HDx, Abbott EnSite Navx, etc.) and is also wired to relay ‘throw 2’. To protect the external electrophysiologic mapping system, the input NO 2 is connected to system ground. The input NO 1 can be alternately connected to positive or negative of the electroporation generator input, depending on the implementation. In some embodiments, another layer of relays can be added to the circuitry to allow for selective software configuration of the polarity for each individual electrode. All of the relays can be controlled by an internal integrated control system connected through an i2c bus.

FIG. 3 depicts a basket catheter 300 having a plurality of electrodes 305 positioned on a plurality of splines 310 in accordance with an illustrative embodiment. FIG. 3 also depicts the polarity of the electrodes positioned on the splines. In the embodiment shown, the basket catheter 300 has 8 splines 310, with a plurality of electrodes 305 on each spline 310, and an electrode spacing of 7 mm between electrodes 305. For illustrative purposes, electrodes 305 and their pluralities are depicted on three of the spines. However, it is to be understood that the electrodes can be similarly positioned on each of the splines 310 included in the basket catheter 300. In an illustrative embodiment, each of the splines 310 can include 8 electrodes 305. Alternatively, a different number of electrodes may be used on each spine, such as 4, 6, 7, 10, 12, etc. In another alternative embodiment, a different number of splines may be used, such as 6, 9, 10, 12, 15, etc. As one example, in some embodiments, the basket catheter can include additional splines and fewer electrodes per spline (e.g. if there are only 4 electrodes per spline, then increasing splines and adjusting the energy density can improve coverage of the system). Similarly, in some embodiments, a different spacing between electrodes can be used, such as 5 mm, 6 mm, 8 mm, 10 mm, etc. Variations of the basket catheter 300 are described in more detail below.

As shown in FIG. 3, longitudinally along each catheter spline 310 the polarity of the electrodes 305 alternates between positive and negative poles. As a result, when the catheter is in its sheath 315, opposite polarities do not come in contact with each other. This strategy also helps prevent shorting when the basket catheter 300 is partially or fully deployed within the atrium. In an illustrative embodiment, the controller device 100 described with reference to FIG. 1 can include software that is able to remotely configure the polarity each electrode 305 as positive or negative polarity for electroporation, as grounded, and/or for use in electrogram measurement. In such an embodiment, once expanded, the polarities of electrodes 305 on adjacent splines can be opposite one another to help induce current flow between splines. For example, in such an embodiment, the first electrode on a first spline can be positive and the first electrode on a second spline (adjacent to the first spline) can be negative. Similarly, the second electrode on the first spline can be negative and the second electrode on the second spline can be positive, and so on. Having the oppositely polarized electrodes across from one another on adjacent splines can increase the ability for current to flow across (i.e., between) splines such that the basket catheter is able to cover a larger area more effectively for purposes of mapping and electroporation. This increased tissue coverage improves both the electroporation and the amount of data that can be detected by the system.

In an illustrative embodiment, the proposed basket catheter 300 can have a shape this is similar or identical to that of the Topera FIRMap 50 catheter. The basket catheter is designed to be spherical or almost spherical in shape (when expanded), with its proximal and distal spline curvature consistent along its length to encourage contact of electrodes. The diameter of the basket catheter 300 can be 40 mm, 50 mm, 60 mm, 70 mm, 80 mm, etc. when the basket is spherical. Alternatively, a different shape and/or size may be used for the basket catheter 300.

In an illustrative embodiment, each electrode 305 on the basket catheter 300 includes an internal insulated wire that is able to sustain at least 10 electroporation sets with each set at a maximum of: 25 pulses at 10 mS at 350V with a duty cycle of 3.3%, without degradation of performance. Alternatively, the electrode wiring can be designed to sustain fewer or additional electroporation sets and/or at different electroporation settings. In another illustrative embodiment, the materials from the components of the basket catheter 300 are bio-compatible to avoid irritation/allergy. In some embodiments, the external catheter sheath 315 does not adhere to the vessel, even under extreme internal heat overload. Additionally, the catheter sheath 315 is designed to be flexible enough to manipulate (e.g., similar to the Topera FIRMap 50). In another illustrative embodiment, the basket catheter 300 is designed such that the distal end of the basket does not protrude and interfere with placement of the distal end against the myocardium.

Catheter electrode spacing has many considerations. For example, with respect to the rigidity/flexibility of splines 310, the splines 310 are designed to be firm enough to assert sufficient electrode contact pressure to the interior atrium wall. Regarding the number of electrodes per spline, as discussed above, 8 electrodes per spline can be used, although this may vary to maintain desired energy densities (V/mm) for the system. Regarding placement of electrodes, the basket catheter 300 includes constant electrode spacing down the spline, and the electrode spacing can be the same on every spline. As also discussed, the electrode spacing can be 7 mm, but other values may be used in alternative embodiments, such as 5 mm, 8 mm, 10 mm, 12 mm, etc. Each electrode 305 on the basket catheter 300 can be the same size such that all electrodes 305 are consistent with one another in length and width. Alternatively, different sizes may be used for different electrodes. In one embodiment, each electrode 305 can have a size of 2.125 mm length×0.075 mm width. In alternative embodiments, different lengths and/or widths may be used for the electrodes 305. Regarding the length of splines 310 (when straightened—non-curved, function of basket size), the spline length can vary between −80 mm and 109.4 mm, in one embodiment. Alternatively, different sizes may be used such as 70 mm, 115 mm, 120 mm, etc.

The electroporation voltage passed through the electrodes 305 is typically 200 V. However, the voltage applied is a variable that can change based upon electrode spacing. As an example, in the Topera FIRMap 50 the spacing is 7 mm to maintain energy densities (V/mm), while the Topera FIRMap 70 has 12 mm spacing. Therefore the voltage may be increased to as much as 343V. The electroporation pulse width is typically 10 mS, although other values can be used. The electroporation duty cycle is a function of heart rate. In some embodiments, the system uses a duty cycle (aka dwell) ranging from 1% to 3.3% (based on 10 mS electroporation and pacing rates 1000 mS to 300 mS respectively). The system can also account for tissue impedance, which is a function of space between electrodes. Also, the electroporation current can be a function of impedance and electroporation voltage. With respect to actual use of the basket catheter, experiments have demonstrated that the same catheter can be used for a minimum of 240 electroporation's (4 experiments, 6 sites each, 10 pulses) at 200V and a duty cycle of 0.01% (10 mS/1000 mS).

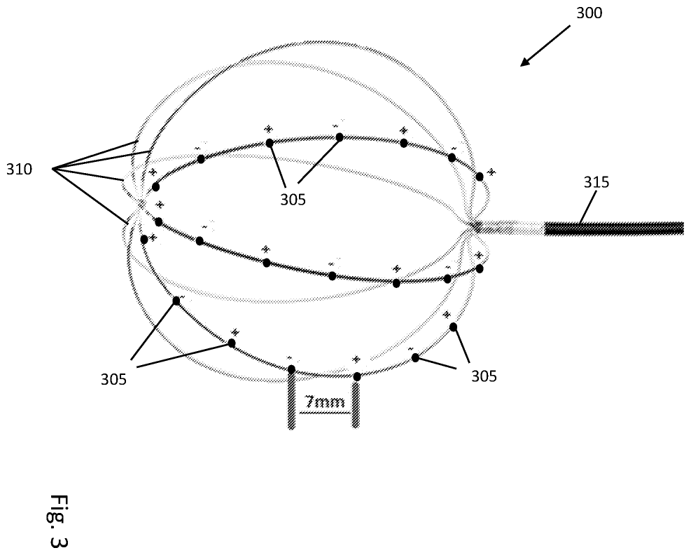

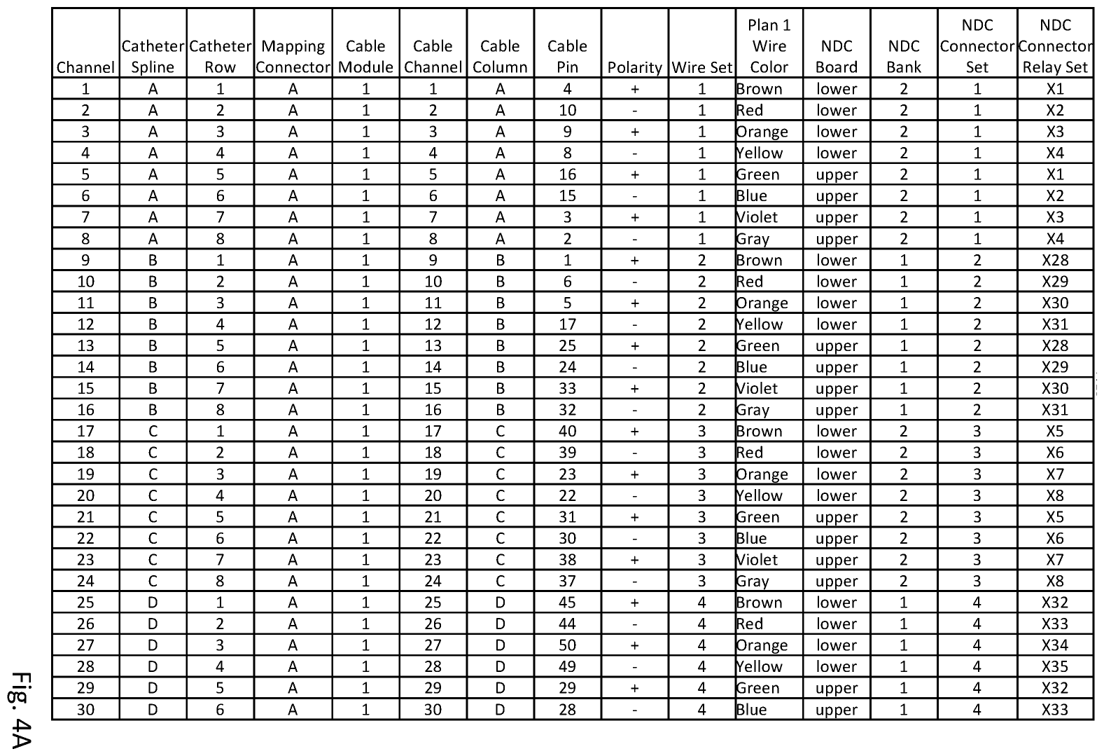

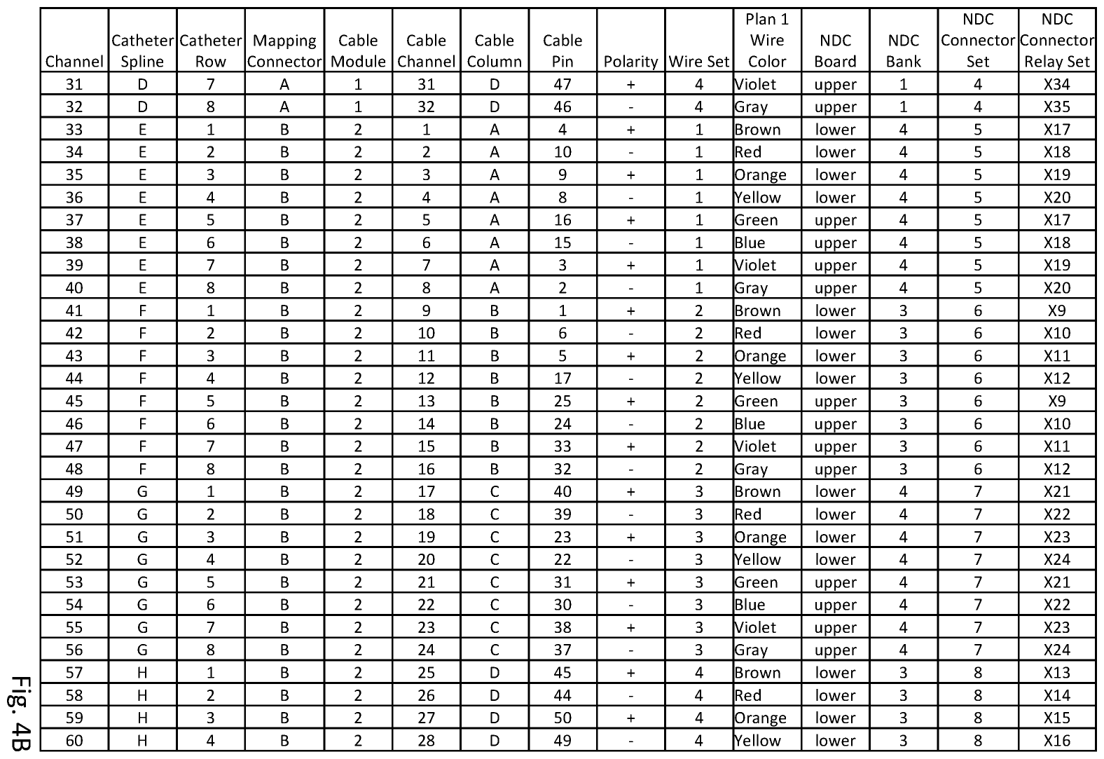

FIG. 4 is an embedded EPEL interface cabling spreadsheet that documents the wiring for each catheter electrode, the mapping system, and the electroporation generator. Specifically, FIG. 4A shows the wiring for catheter splines A, B, and C, and part of spline D in accordance with an illustrative embodiment. FIG. 4B shows the wiring for the remaining portion of catheter spline D, catheter splines E, F, and G, and part of spline H in accordance with an illustrative embodiment. FIG. 4C shows the wiring for the remaining portion of catheter spline H in accordance with an illustrative embodiment. In alternative embodiments, a different wiring configuration may be used for any of the electrodes and/or a different number of splines/electrodes may be used.

As discussed, a computer can be used to implement any of the operations described herein. The computer can have a memory to store code and other computer-readable instructions. A processor of the computer executes the instructions to perform the operations described herein. The computer can also include a user interface such a user can interact with and control the computer, a transceiver for communicating remotely with other computers and/or devices, an operating system to control the computer, etc.

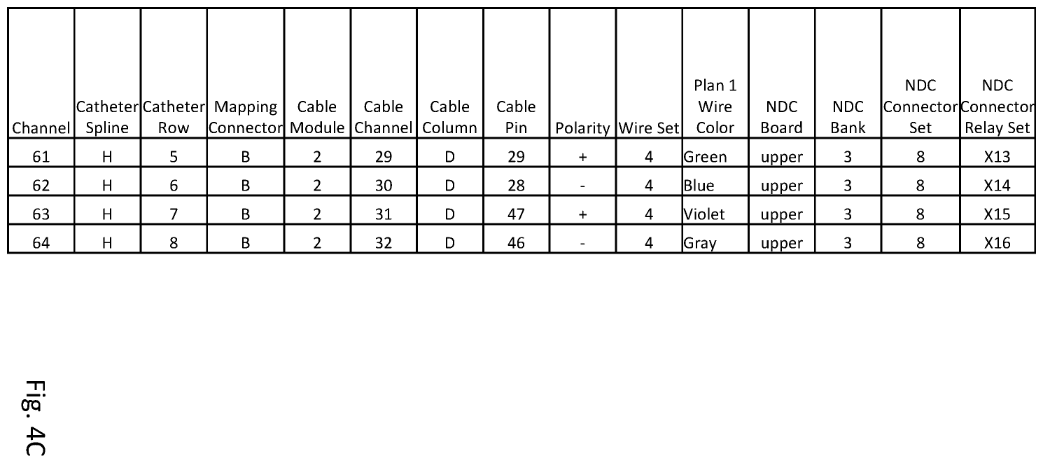

FIG. 5 depicts a computing system 500 in direct or indirect communication with a network 535 in accordance with an illustrative embodiment. The computing system 500 includes a processor 505, an operating system 510, a memory 515, a display 517, an input/output (I/O) system 520, a network interface 525, and a control application 530. In alternative embodiments, the computing system 500 may include fewer, additional, and/or different components. The components of the computing device 500 communicate with one another via one or more buses or any other interconnect system. In an illustrative embodiment, the computing system 500 and any of its functionality can be incorporated as part of the controller device 100 described with respect to FIG. 1.

The processor 505 of the computing system 500 can be in electrical communication with and used to perform any of the operations described herein, such as gathering data (e.g., electroporation execution data, whether the electroporation was successful, mapping data, pacing data, etc.), processing the gathered data, displaying data to a user on the display 517, controlling external systems (e.g., a basket catheter 540, an electroporation generator 545, an external pacing unit 550, a mapping system, etc.), etc. The processor 505 can be any type of computer processor known in the art, and can include a plurality of processors and/or a plurality of processing cores. The processor 505 can include a controller, a microcontroller, an audio processor, a graphics processing unit, a hardware accelerator, a digital signal processor, etc. Additionally, the processor 505 may be implemented as a complex instruction set computer processor, a reduced instruction set computer processor, an x86 instruction set computer processor, etc. The processor 505 is used to run the operating system 510, which can be any type of operating system.

The operating system 510 is stored in the memory 515, which is also used to store programs, algorithms, network and communications data, peripheral component data, the control application 530, and other operating instructions. The memory 515 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc.

The I/O system 520, or user interface, is the framework which enables users (and peripheral devices) to interact with the computing system 500. The I/O system 520 can include one or more keys or a keyboard, one or more buttons, a speaker, a microphone, etc. The I/O system 517 can also control the display 517 such that the user is able to view and control electroporation data, electroporation success data (e.g., a success count), mapping data, pacing data, system settings, etc. The I/O system 520 allows the user to interact with and control the computing system 500. The I/O system 520 also includes circuitry and a bus structure to interface with and control peripheral computing components such as one or more power sources, the basket catheter 540, the electroporation generator 545, the external pacing unit 550, a mapping system, an agent delivery system, etc. In some embodiments, instead of using external systems, a pacing unit, an electroporation generator, a mapping system, and/or an agent delivery system can be incorporated into the computing system 500. The display 517 can be a touch screen display in some embodiments, and can utilize any type of display technology such as LED, LCD, etc.

The network interface 525 includes transceiver circuitry that allows the computing system 500 to transmit and receive data to/from other devices such as user device(s), remote computing systems, servers, websites, etc. The network interface 525 enables communication through the network 535, which can be one or more communication networks. The network 535 can include a cable network, a fiber network, a cellular network, a wi-fi network, a landline telephone network, a microwave network, a satellite network, etc. The network interface 525 also includes circuitry to allow device-to-device communication such as near field communication (NFC), Bluetooth® communication, etc. In alternative embodiments, the computing system 500 may be a standalone system that does not connect to the network 535.

The control application 530 can include hardware, software, and algorithms (e.g., in the form of computer-readable instructions) which, upon activation or execution by the processor 505, performs any of the various operations described herein such as receiving electroporation data, processing electroporation data, determining electroporation success, maintaining an overall electroporation count, maintaining a count of successful electroporations, processing and implementing received system settings and commands (pulse duration, pulse frequency, voltage to be used, duty cycle, etc.), controlling delivery on an agent for the electroporation, generating and/or displaying mapping and other data, controlling the basket catheter 540, controlling the electroporation generator 545, controlling the external pacing unit 550, etc. In some embodiments, the control application 530 can switch between a mapping mode and an electroporation mode such that a user is able to make a comparison of the tissue before and after electroporation such that the effect of the electroporation can be readily determined/viewed. The comparison can also be sued to determine whether the electroporation was successful. As shown, both the external pacing unit 550 and the electroporation generator 545 are connected to the basket catheter 540. The control application 530 can utilize the processor 505 and/or the memory 515 as discussed above.

In an illustrative embodiment, the control application 530 can control the basket catheter 540 and the electroporation generator 545 such that electroporation and/or data collection occurs both along splines of the basket catheter and between adjacent splines of the basket catheter. Thus, instead of being able to cover just an area that is in contact with the splines, the system is able to cover an entire area encompassed by the catheter because of the current that is able to run between adjacent splines of the catheter. Specifically, electricity flows on a current path between oppositely charged electrodes on adjacent splines. For example, current can flow from a first (e.g., positive) electrode on a first spline to a second (e.g., negative) electrode on a second spline that is adjacent to the first spline. Similarly, current can flow between the second electrode on the second spline and a third (e.g., positive) electrode on a third spline that is adjacent to the second spline, and so on to provide full (current path) coverage of the area encompassed by the basket catheter. As discussed, the electricity also flows along each spline (i.e., between the alternating oppositely charged electrodes that are mounted along each spline).

For purposes of electroporation, mapping, pacing, etc., the control application 530 can also control electrical current through the basket catheter in a sequential or simultaneous fashion. For example, the control application 530 can sequentially control electrode activation such that a first portion of the basket catheter is activated prior to a second portion of the basket catheter. The sequential activation can be along splines and/or across splines, as discussed herein. The timing between sequential activations of electrodes can be based on the type of agent being delivered, the desired rate of agent delivery, etc. Simultaneous activation of all (or a subset) of the electrodes can be used, for example, to electroporate or monitor all of the atrium (or other tissue upon which the catheter is placed) or a desired portion of the atrium.

As also discussed, each of the electrodes can be bi-polar and configurable by the control application 530. As such, the control application 530 can be used to individually control the polarity of each electrode, to tie any electrode to ground, for use in electrogram measurement, and/or for any other of the electrode operations described herein. In such an embodiment, the system includes a plurality of bi-polar electrodes to provide maximal configurability for the user.

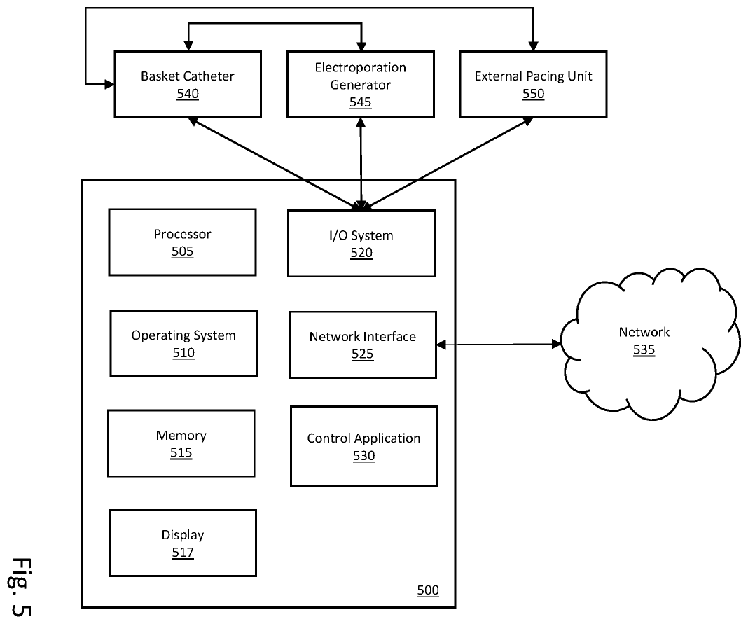

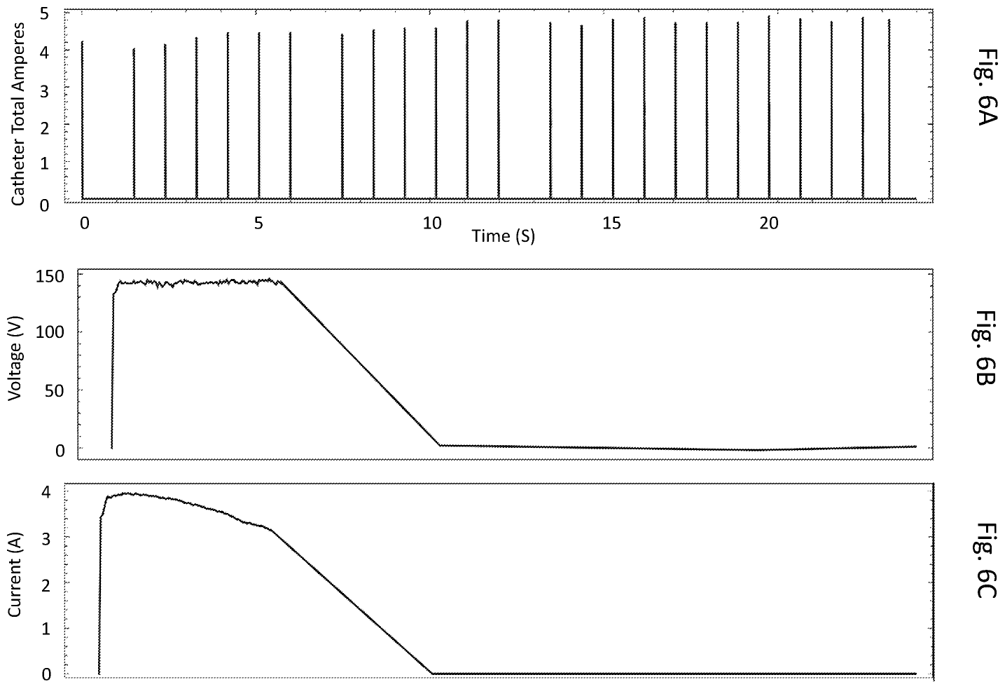

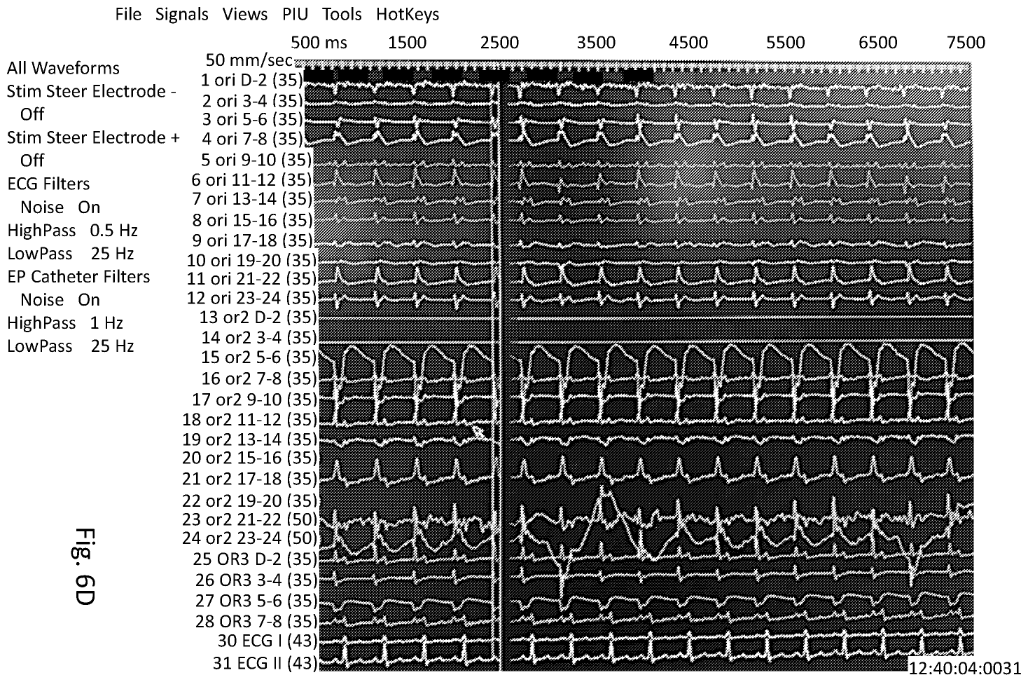

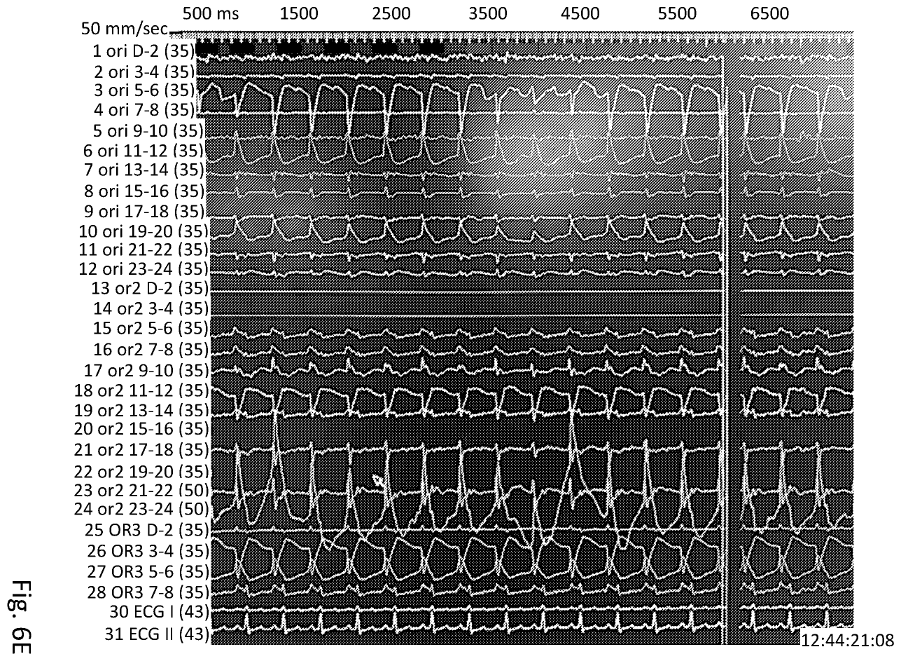

FIG. 6 depicts results of an electroporation process performed on a patient using the proposed system. Specifically, FIG. 6A depicts a pulse train and total current (in Amperes) delivered by an electroporation generator to a catheter across all electrodes in accordance with an illustrative embodiment. In FIG. 6A, each spike represents an electroporation pulse which was programmed to deliver 142V and was 10 mS in duration. Each of the pulses shown in FIG. 6A was controlled to be in sync with a paced beat which occurred during the QRS wave of the ECG. FIG. 6B depicts a pulse from the pulse train of FIG. 6A in accordance with an illustrative embodiment. As shown, the pulse of FIG. 6B measures ˜142V and its duration was 10 mS, shortly after which it decays back to 0 Volts. FIG. 6C depicts the current in a single electrode on a spline of the catheter in accordance with an illustrative embodiment. As shown, the duration of the current is again approximately 10 mS, and the current decays back to 0 Amperes shortly thereafter.

Electrograms were taken both prior to and subsequent to the electroporation performed with reference to FIGS. 6A-6C. FIG. 6D depicts an electrogram taken prior to electroporation using the proposed system in accordance with an illustrative embodiment. FIG. 6E depicts an electrogram taken subsequent to electroporation using the proposed system in accordance with an illustrative embodiment. The images from FIGS. 6D and 6E are taken with the proposed catheter in the Left Atrial Appendage (LAA) of the heart of the patient. These images illustrate that the electrical activity in the heart of the patient is not substantially different after electroporation using the proposed method.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

1. A system for performing integrated mapping and electroporation, the system comprising:
a basket catheter that includes a plurality of splines and a plurality of electrodes mounted to each spline in the plurality of splines; and
a controller device connected to the basket catheter, wherein the controller device includes a processor configured to activate at least a subset of electrodes to perform electroporation, wherein activation of at least the subset of electrodes generates current paths between electrodes on a given spline and between electrodes on adjacent splines.

2. The system of claim 1, wherein the plurality of electrodes alternate between a positive electrode and a negative electrode along a length of a spline.

3. The system of claim 1, wherein the controller device is further configured to:
receive a pacing signal for a heart that is in contact with the basket catheter; and
synchronize the electroporation with the pacing signal to prevent ventricle defibrillation.

4. The system of claim 3, further comprising a pacing unit that is in communication with the controller device, wherein the pacing signal is received from the pacing unit.

5. The system of claim 1, further comprising an electroporation generator that is in communication with the controller device and connected to the basket catheter, wherein the controller device controls the electroporation generator to deliver voltages to the subset of electrodes.

6. The system of claim 1, wherein the controller device is further configured to receive mapping data from the subset of electrodes.

7. The system of claim 6, wherein the controller device is configured to alternate the basket catheter between a mapping mode to obtain the mapping data from the subset of electrodes and an electroporation mode to perform the electroporation by the subset of electrodes.

7. (canceled)

8. The system of claim 1, wherein the controller device is further configured to control agent delivery in conjunction with the electroporation, wherein the agent delivery comprises in-vivo delivery of one or more genes into a heart of a patient.

9. The system of claim 1, wherein the controller device is further configured to determine whether the electroporation is successful.

10. The system of claim 9, wherein the controller device is configured to maintain a count of successful electroporations using the basket catheter.

11. The system of claim 1, wherein at least the subset of electrodes comprises all of the electrodes on the basket catheter to maximize a coverage area of the basket catheter.

12. The system of claim 1, wherein activation of the subset of electrodes is performed sequentially such that one or more first electrodes is activated prior to one or more second electrodes.

13. The system of claim 1, wherein activation of the subset of electrodes is performed simultaneously.

14. The system of claim 1, wherein the plurality of electrodes are bi-polar, and wherein the controller device is configured to set a polarity of each electrode as negative or positive.

15. A basket catheter for use in performing integrated mapping and electroporation, wherein the basket catheter comprises:
a sheath;
a plurality of splines mounted to the sheath;
a plurality of electrodes mounted to each spline in the plurality of splines, wherein the plurality of electrodes alternate between a positive electrode and a negative electrode along a length of each spline, and wherein the plurality of splines are configured such that current flow occurs both between electrodes along a given spline and between electrodes on adjacent splines.

16. The basket catheter of claim 15, wherein each electrode in the plurality of electrodes is bi-polar such that each electrode can transition to a positive electrode or a negative electrode.

17. The basket catheter of claim 15, wherein the plurality of splines comprises eight splines, and wherein the plurality of electrodes mounted to each spline comprises eight electrodes.

18. The basket catheter of claim 15, wherein a spacing between the plurality of electrodes mounted to each spline is seven millimeters.

19. The basket catheter of claim 15, wherein each of the electrodes is individually wired such that each electrode can be controlled independent of any other electrode.

20. The basket catheter of claim 15, wherein each of the electrodes is configured for placement into a mapping mode and an electroporation mode.
